(19) United States
(12) Patent Application Publication
(10) Pub. No.: US 2016/0245649 A1
(43) Pub. Date: Aug. 25, 2016

(54) Castable Sensor Device

(71) Applicant: Navico Holding AS, Egersund (NO)

(72) Inventors: Matthew Laster, Broken Arrow, OK (US); Lucas Dale Steward, Broken Arrow, OK (US)

(21) Appl. No.: 14/628,154

(22) Filed: Feb. 20, 2015

(51) Int. Cl. G01C 13/00; G01N 33/18

(52) U.S. Cl. CPC G01C 13/00; G01N 33/1886

(57) ABSTRACT

Various implementations described herein are directed to a device having at least one environmental sensor configured to generate sensor data at one or more depths of the device in a body of water. The device may be configured to record the sensor data received from the at least one environmental sensor in response to the device being deployed at the one or more depths in the body of water.

### BACKGROUND

This section is intended to provide information to facilitate an understanding of various technologies described herein. As the section's title implies, this is a discussion of related art. That such art is related in no way implies that it is prior art. The related art may or may not be prior art. It should therefore be understood that the statements in this section are to be read in this light, and not as admissions of prior art.

When trolling for fish, environmental related data is useful. Communicating with a device that collects this data may be beneficial to an angler.

### SUMMARY

Described herein are implementations of various technologies for a device having an environmental sensor configured to generate sensor data at one or more depths of the device in a body of water. The device may include a processor and memory having instructions that cause the processor to receive the sensor data from the environmental sensor in response to the device being deployed at the one or more depths in the body of water and record the sensor data.

Described herein are also implementations of various technologies for a castable lure. The castable lure may include a first sensor configured to determine water pressure at one or more depths of the castable lure in a body of water, and generate depth data based on the determined water pressure at the one or more depths of the castable lure in the body of water. The castable lure may include a second sensor configured to generate environmental data corresponding to the depth data at the one or more depths of the castable lure in the body of water. The castable lure may include a computing component configured to record the first sensor data generated by the first sensor and record the second sensor data generated by the second sensor.

Described herein are also implementations of various technologies for a device having a castable housing impervious to water. The device may include one or more environmental sensors configured to generate sensor data at one or more depths in the water. The device may include a processor and memory including instructions that cause the processor to record the sensor data generated by the one or more environmental sensors at the one or more depths in the water, and transmit the sensor data in response to detecting a network.

The above referenced summary section is provided to introduce a selection of concepts in a simplified form that are further described below in the detailed description section. The summary is not intended to identify key features or essential features of the claimed subject matter, nor is it intended to be used to limit the scope of the claimed subject matter. Moreover, the claimed subject matter is not limited to implementations that solve any or all disadvantages noted in any part of this disclosure.

### BRIEF DESCRIPTION OF THE DRAWINGS

Implementations of various techniques will hereafter be described with reference to the accompanying drawings. It should be understood, however, that the accompanying drawings illustrate only the various implementations described herein and are not meant to limit the scope of various techniques described herein.

FIGS. 1A-1C illustrate various views of using a castable sensor device in accordance with various implementations described herein.

FIGS. 2A-2B illustrate various diagrams of sensor systems in accordance with various implementations described herein.

FIGS. 3-4 illustrate process flows of methods for using a castable sensor device in accordance with various implementations described herein.

FIG. 5 illustrates a schematic of a marine electronics device in accordance with various implementations described herein.

### DETAILED DESCRIPTION

Various implementations described herein are directed to using a castable sensor device. In some implementations, various techniques described herein refer to environmental sensor technology for detecting various environmental conditions in a body of water. For instance, one or more environmental sensors may be attached to a fishing line and casted in a body of water to detect various environmental conditions at a surface outside of the water as well as in the water during ascent and descent through the water. Once deployed, the one or more environmental sensors may generate and transmit environmental data related to detected environmental conditions to an onboard computing device for display by, e.g., overlaying various environmental data on chart and sonar images. In some instances, various environmental data may include current levels of environmental conditions at particular depths, upper and lower boundary levels at particular depths, average levels through a water column, and any changes that occur throughout sensor use during a particular time period or interval. The castable sensor device may include the one or more environmental sensors and may be used in either saltwater and/or freshwater. The castable sensor device may be attached to a cast line or attached as a fishing lure. These various techniques may provide an angler desirable environmental condition data of water at various depths in a water column for adjusting location, position, and/or depth of fishing lures while fishing and before moving through a desirable area where fish may be running or holding.

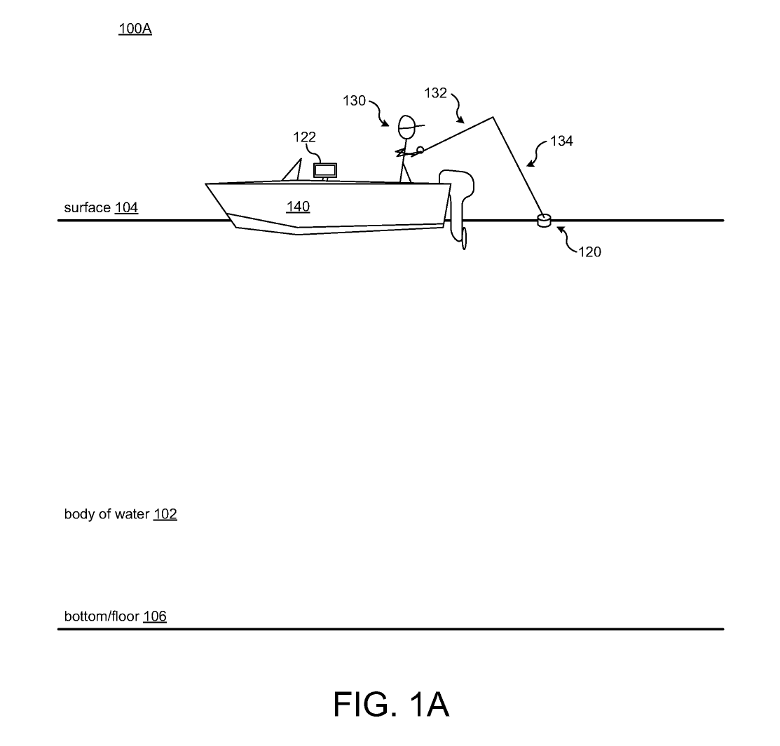

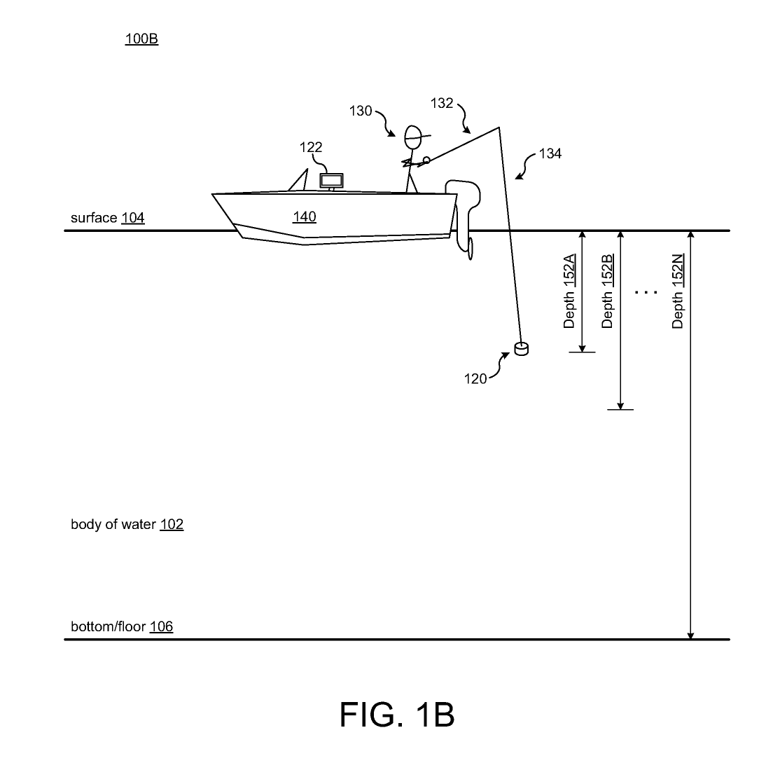

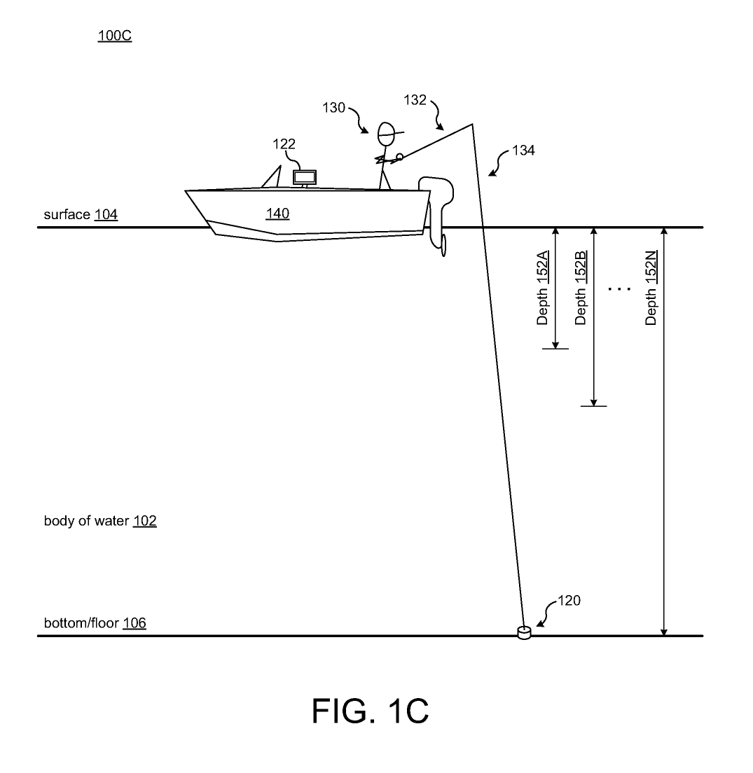

Various implementations of using a castable sensor device will now be described in reference to FIGS. 1A-5.

FIGS. 1A-1C illustrate various views of using a castable sensor device 120 in accordance with various implementations described herein. In particular, FIG. 1A illustrates a view of using the castable sensor device 120 at a surface 104 (e.g., zero depth) of a body of water 102, FIG. 1B illustrates another view of using the castable sensor device 120 at a depth 152A, and FIG. 1C illustrates another view of using the castable sensor device 120 at another depth 152N, such as near a bottom or floor 106 of the body of water 106.

In reference to FIG. 1A, the castable sensor device 120 may include one or more environmental sensors configured to generate sensor data at one or more depths of the device 120 in a column of water in the body of water 102, such as, e.g., ocean, sea, gulf, lake, river, stream, pond, etc. In various implementations, the one or more environmental sensors may be configured to detect levels of environmental content, concentrations, and/or characteristics of the body of water 102 at the surface 104 and/or at various depths beneath the surface 104. For instance, the detectable environmental characteristics may include one or more of pressure (e.g., atmospheric pressure, water pressure, etc.), chlorophyll concentration, oxygen concentration, temperature, light intensity, electrolyte concentration (e.g., salt content), acidity, etc.

In some implementations, the castable sensor device 120 may include various computing, processing, and storage components, such as, e.g., at least one processor and memory. The memory may include instructions that cause the processor to receive sensor data from the one or more environmental sensors after deployment of the castable sensor device 120 in the body of water 102 and record the sensor data received from the one or more environmental sensors at one or more depths of the castable sensor device 120 in the body of water 102. In some instances, the sensor data may be received and/or recorded at one or more depths during vertical movement (e.g., ascent and/or descent) of the castable sensor device 120 in the body of water 102. In some other instances, the sensor data may be received and/or recorded at one or more depths while the castable sensor device 120 is holding or remains stationary in the body of water 102.

In some implementations, the memory may include instructions that cause the processor to transmit sensor data generated by the one or more environmental sensors and/or recorded by processor. In some instances, the castable sensor device 120 may include a network interface, such as, e.g., a transmitter, transceiver, etc., to transmit sensor data to a computing device 122, such as, e.g., a marine electronics device, multi-function display (MFD), smart phone, computer, laptop, tablet, etc. The computing device 122 may be configured to store, record, and/or log sensor data received from the castable sensor device 120. Further, the computing device 122 may be configured to display sensor data and/or various images associated with sensor data received from the castable sensor device 120 on a display component, such as, e.g., a monitor or other computer display.

In various instances, transmission of sensor data between the castable sensor device 120 and the computing device 122 may occur via a wired or wireless communication network. Further, in some instances, transmission of sensor data between the castable sensor device 120 and the computing device 122 may occur when the castable sensor device 120 surfaces from the body of water 102. In some other instances, transmission of sensor data between the castable sensor device 120 and the computing device 122 may occur while the castable sensor device 120 is submerged (or during submersion) underneath the surface 104 of the body of water 102 via one or more underwater wired or wireless communication channels.

The one or more environmental sensors may be configured to periodically generate sensor data at pre-determined time intervals and/or at pre-determined depth intervals. For instance, the pre-determined time intervals may refer to generating sensor data for each unit of time, such as, e.g., seconds, minutes, etc. In another instance, the pre-determined depth intervals may refer to generating sensor data for each unit of depth, such as, e.g., inches, feet, meters, etc. In some other instances, since depth may relate to pressure, the pre-determined depth intervals may refer to generating sensor data for each unit of change in water pressure, such as, e.g., Pascal's, pounds per square inch (psi), bars, atmospheres, etc.

During trolling for fish, the castable sensor device 120 including the one or more environmental sensors may be used to detect, determine, and/or identify levels of environmental content, concentrations, and/or characteristics of the body of water 102 at the surface 104 and/or at various depths beneath the surface 104. In various instances, sensor data generated by the one or more environmental sensors may be used to assist a user 130 (e.g., boat pilot, fisherman, angler, etc.) with locating or finding a desirable place to fish in the body of water 102. Generally, environmental conditions in the body of water 102 may affect where fish and/or schools of fish are holding. Therefore, in some instances, knowing the environmental conditions in the body of water 102 beneath a vessel 140 at various depths may be beneficial to the user 130. For instance, during trolling, the castable sensor device 120 may be coupled to a casting device, such as a rod 132 (e.g., a fishing rod or pole), via a line 134 (e.g., a fishing line). The rod 132 may be configured for casting the castable sensor device 120 by the user 130. As shown in FIG. 1A, the user 130 may cast the castable sensor device 120 into the body of water 102 proximate to a stern of the vessel 140, while the user 130 is positioned within the vessel 140. However, in various other instances, the user 130 may cast the castable sensor device 120 from anywhere on the vessel 140, while the user 130 is positioned within the vessel 140.

In various implementations, deployment of the castable sensor device 120 in the body of water 102 may include casting the castable sensor device 120 in the body of water 102 by the user 130, as shown in FIG. 1A. Therefore, in various instances, the castable sensor device 120 may include a castable housing, such as a waterproof housing that is impervious to water. In some instances, the castable sensor device 120 may include a castable lure or fishing bait. Further, in some instances, the one or more environmental sensors of the castable sensor device 120 may be encapsulated within the waterproof housing that is impervious to water so as to protect the one or more environmental sensors from water damage. In some other implementations, each of the one or more environmental sensors may be encased in a waterproof material that is impervious to water for protection from water damage.

In one implementation, the castable sensor device 120 may be configured as a castable lure having one or more environmental sensors. For instance, the castable lure may include a first sensor configured to determine water pressure at one or more depths of the castable lure in water (e.g., body of water 102). In this instance, the first sensor may be configured to further generate depth data based on the determined water pressure at one or more depths of the castable lure in the body of water 102. Further, the castable lure may include at least one other sensor (e.g., a second sensor) configured to generate various types of environmental data (e.g., one or more of chlorophyll concentration, oxygen concentration, temperature, light intensity, electrolyte concentration, salt content, acidity, etc.) corresponding to the depth data at the one or more depths of the castable lure in the body of water 102. Further, in some instances, the castable lure may include some type of computing component configured to record the first sensor data generated by the first sensor and the second sensor data generated by the second sensor.

In various implementations, the castable lure may be in the form of one or more environmental sensors being added or integrated to/with a fishing bait, whereby the environmental sensors are configured to record various depths that the fishing bait reaches on a cast or troll and/or also to collect various environmental conditions (e.g., sensed levels) at those various depths. The castable lure may be configured to output environmental sensor data and information to a computing device for display. This output may be transmitted via wired or wireless communication. The displayed data and information may be for a single cast, multiple casts, and/or data combined from multiple different casts to create a depth and temperature map of an area on a body of water. Various types of fishing bait, such as, e.g., a crank bait, may be used with this castable lure technique. Further, in some instances, this castable lure technique may assist anglers and fishermen to know various lure depths on multiple cast trolls so as to assist with making changes to the manner in which reeling or holding a rod angle may be used to dial in on a particular depth that bait are running or holding. This information may assist with looking at fish depth on fish finder sonar and wanting bait fish to pass by at a same level.

In reference to FIGS. 1B-1C, the castable sensor device 120 may include one or more environmental sensors configured to generate sensor data at one or more depths 152A, 152B, . . . , 152N of the device 120 in a column of water beneath the surface 104 of the body of water 102. During trolling, the castable sensor device 120 including the one or more environmental sensors may be used to detect, determine, and/or identify levels of environmental content, concentrations, and/or characteristics of the water in the body of water 102 at the surface 104 and/or at the various depths 152A, 152B, . . . , 152N beneath the surface 104 and/or beneath the vessel 140.

For instance, as shown in FIG. 1B, the one or more environmental sensors of the castable sensor device 120 may be configured to generate sensor data at the depth 152A of the device 120 in a column of water beneath the surface 104 of the body of water 102. Further, as shown in FIG. 1C, the one or more environmental sensors of the castable sensor device 120 may be configured to generate sensor data at the depth 152N (e.g., near the bottom or floor 106 of the body of water 102) of the device 120 in a column of water beneath the surface 104 of the body of water 102.

In some instances, the surface 104 of the body of water 102 may be referred to as zero depth or an upper boundary of depth, and the bottom or floor 106 of the body of water 102 may be referred to as a lower boundary of depth. In some instances, the various depths 152A, 152B, . . . , 152N may refer to various depth positions, including the upper and lower boundaries, in a water column between the surface 104 and the floor 106 of the body of water 102. Therefore, in various instances, each of the one or more various depths 152A, 152B, . . . , 152N may refer to any depth position in the water column between the surface 104 and the floor 106 of the body of water 102.

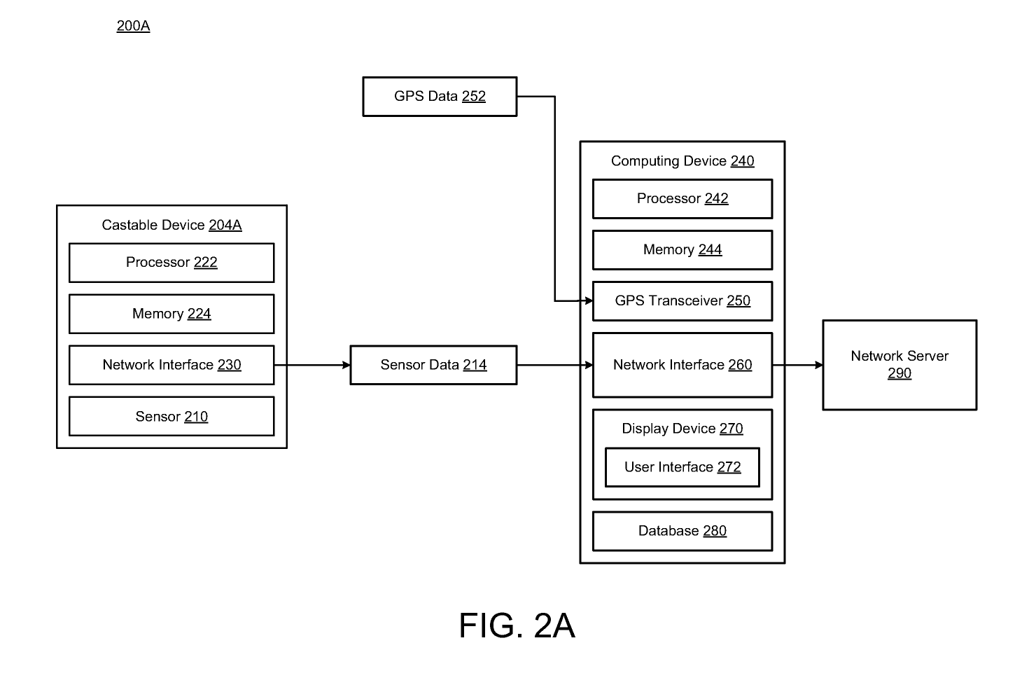

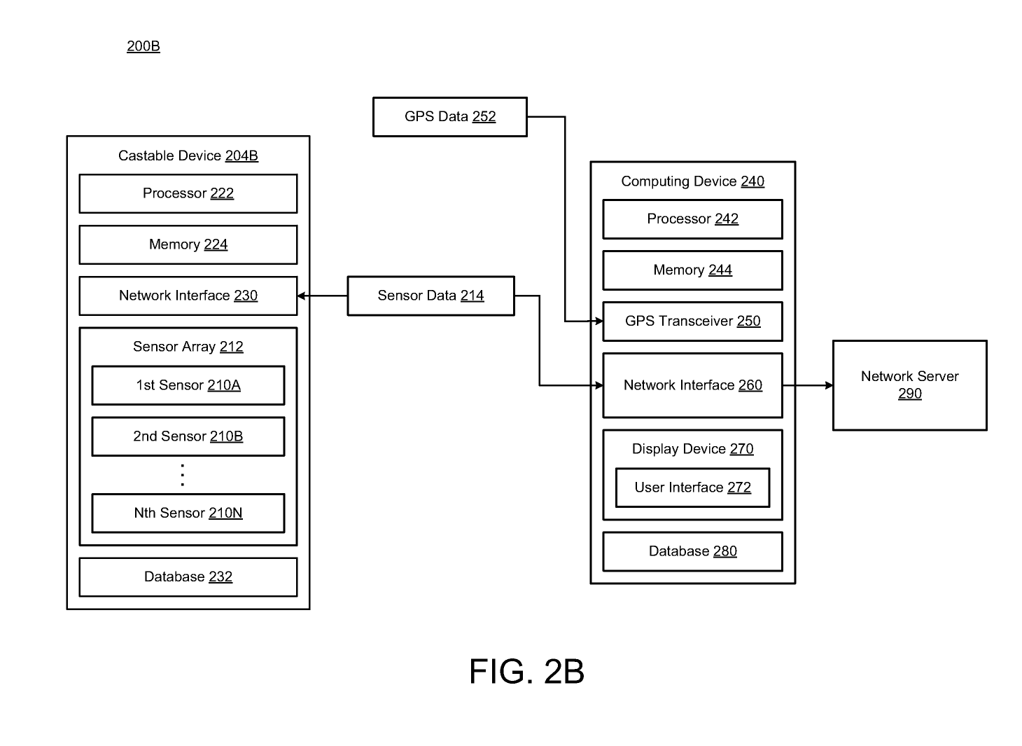

FIGS. 2A-2B illustrate various diagrams of sensor systems 200A, 200B in accordance with various implementations described herein. In particular, FIG. 2A illustrates a diagram of the sensor system 200A using a castable device 204A with at least one environmental sensor 210, and FIG. 2B illustrates a diagram of the sensor system 200B using a castable device 204B with a sensor array 212 having multiple environmental sensors 210A, 210B, . . . , 210N. In various implementations, the sensor array 212 may include any number of environmental sensors 210A, 210B, . . . , 210N, including one, two, or more than two environmental sensors.

In reference to FIG. 2A, the sensor system 200A may include the castable device 204A having the at least one environmental sensor 210, a computing device 240, and a network server 290. In some implementations, the castable device 204A may be configured to generate and transmit sensor data 214 to the computing device 240 via a wired or wireless network, and the computing device 240 may be configured to transmit or upload the sensor data received from the castable device 204A to the network server 290 via a wired or wireless network.

The castable device 204A may include the at least one environmental sensor 210, at least one processor 222, memory 224, and a network interface 230. The at least one environmental sensor 210 may be configured to generate sensor data at one or more depths of the castable device 204A in water (e.g., body of water 102). The at least one environmental sensor 210 may be configured to detect levels of environmental content, concentrations, and/or characteristics of water at a surface and/or at various depths beneath the surface. For instance, the detectable environmental characteristics may include one or more of pressure (e.g., atmospheric pressure, water pressure, etc.), chlorophyll concentration, oxygen concentration, temperature, light intensity, electrolyte concentration (e.g., salt content), acidity, etc.

The castable device 204A may include the processor 222 and the memory 224 having instructions that cause the processor 222 to receive sensor data from the at least one environmental sensor 210 and record the sensor data received from the at least one environmental sensor 210 at one or more depths (e.g., at a surface and/or during vertical movement) of the castable device 204A in a column of water. In some instances, vertical movement refers to ascent and/or descent of the castable device 204A in a column of water. In some other instances, sensor data may be generated by the at least one environmental sensor 210 and recorded while the castable device 204A is holding or remains stationary in a column of water.

The castable device 204A may include the network interface 230, and the memory 224 may include instructions that cause the processor 222 to transmit sensor data 214 generated by the at least one environmental sensor 210 and/or recorded by processor 222. In some instances, the network interface 230 may include a transmitter or transceiver configured to transmit sensor data to the computing device 240, such as, e.g., a marine electronics device, multi-function display (MFD), smart phone, computer, laptop, tablet, etc. In various instances, transmission of sensor data 214 between the castable device 204A and the computing device 240 may occur via a wired or wireless network. Further, in some instances, transmission of sensor data between the castable device 204A and the computing device 240 may occur when the castable device 204A surfaces from water. Further, in some instances, transmission of sensor data may occur in response to detecting a network, such as a wired or wireless network. In some other instances, transmission of sensor data between the castable device 204A and the computing device 240 may occur while the castable device 204A is submerged in water via an underwater wired or wireless channel.

In some implementations, the castable device 204A may be configured to transmit sensor data to the computing device 240 through the body of water 102 with water as a transmission channel or medium for wireless communication between the castable device 204A and the computing device 240. Therefore, the network interface 230 of the castable device 204 may include a transmitter or transceiver configured to transmit sensor data to the network interface 260 of the computing device 240 through the body of water 102 with water as a transmission channel or medium for wireless communication between the castable device 204A and the computing device 240.

In various implementations, the castable device 204A may be deployed in water (e.g., in the body of water 102), which may include casting the castable device 204A in water by a user. Therefore, the castable device 204A may include a waterproof housing that is impervious to water. In some instances, the castable device 204A may include a castable lure or fishing bait. Further, in some instances, the at least one environmental sensor 210 and other components of the castable device 120 may be encapsulated within the waterproof housing for protection from water damage.

In some implementations, the computing device 240 may be configured to receive sensor data 214 from the castable device 204A over a wired or wireless network via a network interface 260. The computing device 240 may include a processor 242 and memory 244 having instructions that cause the processor 242 to the sensor data 214 and/or display images associated with the sensor data 214 on a display component or device 270. Further, the computing device 240 may be configured to create/generate sensor data logs associated with the sensor data 214. The computing device 240 may be configured to store, record, and/or log the sensor data 214 and/or sensor data logs in one or more databases (e.g., database 280). Further, the computing device 240 may be configured to upload the sensor data 214 and/or sonar logs to the network server 290, such as, e.g., a cloud server or other network server, via the network interface 260.

In some implementations, the computing device 240 may be configured to store and record multiple sensor data logs and create/generate one or more sensor data maps of a body of water therefrom. For instance, the computing device 240 may be configured to create/generate one or more maps by stitching, combining, and/or joining multiple sensor data logs together. Further, in some instances, the computing device 240 may be configured to receive geo-coordinate data, such as global positioning system data (i.e., GPS data 252), via a GPS transceiver 250 and associate the received GPS data 252 to the sensor data 214, sensor data logs, and/or sensor data maps at any time, including prior to upload to the network server 290.

In various implementations, the computing device 240 may be configured as a special purpose machine for interfacing with a castable device 204A having at least one environmental sensor 210. Further, the computing device 240 may include various standard elements and/or components, including the at least one processor 242, the memory 244 (e.g., non-transitory computer-readable storage medium), at least one database 280, power, peripherals, and various other computing components that may not be specifically shown in FIG. 2A. Further, the computing device 240 may include the display device 270 (e.g., a monitor or other computer display) that may be used to provide a user interface (UI) 272, including a graphical user interface (GUI). In FIG. 2A, the display 270 is shown as an incorporated part of the computing device 240; however, the display 270 may be implemented as a separate component. Further, the UI 272 may be used to receive one or more preferences from a user of the display device 270 for managing or utilizing the sensor system 200A, including interfacing with the castable device 204A and the one or more environmental sensors 210. As such, a user may setup desired behavior of the sensor system 200A and/or the one or more environmental sensors 210 via user-selected preferences using the UI 272 associated with the display device 270. Various elements and/or components of the system 200A that may be useful for the purpose of implementing the system 200A may be added, included, and/or interchanged, in manner as described herein.

In reference to FIG. 2B, the sensor system 200B may include the castable device 204B having the sensor array 212 with multiple environmental sensors 210A, 210B, . . . , 210N, the computing device 240, and the network server 290. The castable device 204B may be referred to as a castable sensor array. In some implementations, the castable device 204B may be configured to generate and transmit sensor data 214 (including, e.g., sensor data associated with one or more of the multiple environmental sensors 210A, 210B, . . . , 210N) to the computing device 240 via a wired or wireless network. In some instances, the castable device 204B may be configured to transmit sensor data in response to detecting a network, such as a wired or wireless network or communication channel. Further, in some instances, the computing device 240 may be configured to transmit or upload the sensor data received from the castable device 204A to the network server 290 via a wired or wireless network. FIG. 2B may include various similar elements as shown and described in reference to FIG. 2A.

In various implementations, each of the environmental sensors 210A, 210B, . . . , 210N may be configured to generate sensor data at one or more depths of the castable device 204B in water (e.g., body of water 102). In various instances, each of the environmental sensors 210A, 210B, . . . , 210N may be configured to detect levels of environmental content, concentrations, and/or characteristics of water at a surface and/or at various depths beneath the surface. As described herein, the environmental characteristics may include one or more of pressure (e.g., atmospheric pressure, water pressure, etc.), chlorophyll concentration, oxygen concentration, temperature, light intensity, electrolyte concentration (e.g., salt content), acidity, etc.

Further, in various implementations, each of the environmental sensors 210A, 210B, . . . , 210N may be configured to periodically generate environmental sensor data at pre-determined time intervals and/or at pre-determined depth intervals. For instance, the pre-determined time intervals may refer to generating environmental sensor data for each unit of time, such as, e.g., seconds, minutes, etc. In another instance, the pre-determined depth intervals may refer to generating environmental sensor data for each unit of depth, such as, e.g., inches, feet, meters, etc. In some other instances, the pre-determined depth intervals may refer to generating sensor data for each unit of change in water pressure, such as, e.g., Pascal's, psi, bars, atmospheres, etc.

In various implementations, at least one of the environmental sensors 210A, 210B, . . . , 210N may include a pressure sensor configured to detect, determine, and/or identify pressure at one or more depths of the castable device 204B in water (e.g., a column of water in the body of water 102). The pressure sensor may be configured to generate sensor data including pressure data corresponding to depth data at the one or more depths of the castable device 204B. The pressure sensor may be configured to detect, determine, and/or identify one or more of atmospheric pressure, barometric pressure, water pressure, etc. In some instances, the pressure sensor data may be received and/or recorded at a surface of a column of water. In some instances, the pressure sensor data may be received and/or recorded at one or more depths during vertical movement (e.g., ascent and/or descent) of the castable device 204B in a column of water. In some other instances, the pressure sensor data may be received and/or recorded at one or more depths while the castable device 204B is holding or remains stationary in the column of water.

In some instances, fish may become more or less active when water pressure drops or rises. Thus, an environmental sensor that detects pressure in the atmosphere as well as under the water surface may assist anglers and fishermen with detecting pressure changes to determine when fish may be more or less active. Therefore, the castable device 204B with one or more environmental sensors 210A, 210B, . . . , 210N may assist anglers and fishermen with targeting and learning when and where fish are more or less active to possibly reduce unproductive fishing times.

In various implementations, at least one of the environmental sensors 210A, 210B, . . . , 210N may include a chlorophyll sensor configured to detect, determine, and/or identify chlorophyll concentration at one or more depths of the castable device 204B in a column of water. Further, the chlorophyll sensor may be configured to generate sensor data including chlorophyll concentration data corresponding to depth data at the one or more depths of the castable device 204B. In some instances, the chlorophyll sensor data may be received and/or recorded at a surface of a column of water. In some instances, the chlorophyll sensor data may be received and/or recorded at one or more depths during vertical movement (e.g., ascent and/or descent) of the castable device 204B in a column of water. In some other instances, the chlorophyll sensor data may be received and/or recorded at one or more depths while the castable device 204B is holding or remains stationary in the column of water.

In some instances, smaller bait fish tend to feed on chlorophyll immersed in water, and larger bait fish tend to follow the smaller bait fish around hoping to feed off of them. Larger game fish tend to feed off of the larger bait fish. In these instances, a chlorophyll sensor may assist anglers and fisherman in locating and finding where bait fish may be gathering, and a chlorophyll sensor may assist anglers and fisherman with either catching bait for use on game fish or catching game fish in an area where the chlorophyll is gathering or has gathered. Therefore, the castable device 204B with one or more environmental sensors 210A, 210B, . . . , 210N may assist anglers and fishermen with targeting and learning when and where small bait fish may be gathering.

In various implementations, at least one of the environmental sensors 210A, 210B, . . . , 210N may include an oxygen sensor configured to detect, determine, and/or identify oxygen concentration at one or more depths of the castable device 204B in a column of water. Further, the oxygen sensor may be configured to generate sensor data including oxygen concentration data corresponding to depth data at the one or more depths of the castable device 204B. In some instances, the oxygen sensor data may be received and/or recorded at a surface of a column of water. In some other instances, the oxygen sensor data may be received and/or recorded at one or more depths during vertical movement (e.g., ascent and/or descent) of the castable device 204B in a column of water. In still some other instances, the oxygen sensor data may be received and/or recorded at one or more depths while the castable device 204B is holding or remains stationary in the column of water.

In some instances, fish may become inactive and lethargic in water columns having low oxygen levels. In these instances, searching for higher levels of oxygen in various water columns may assist anglers and fishermen with detecting active fish and productive water depths for fishing. Therefore, the castable device 204B with one or more environmental sensors 210A, 210B, . . . , 210N may assist anglers and fishermen with targeting highly oxygenated waters to possibly increase fishing productivity.

In various implementations, at least one of the environmental sensors 210A, 210B, . . . , 210N may include a temperature sensor configured to detect, determine, and/or identify temperature at one or more depths of the castable device 204B in a column of water. Further, the temperature sensor may be configured to generate sensor data including temperature data corresponding to depth data at the one or more depths of the castable device 204B. In some instances, the temperature sensor data may be received and/or recorded at a surface of a column of water. In other instances, the temperature sensor data may be received and/or recorded at one or more depths during vertical movement (e.g., ascent and/or descent) of the castable device 204B in a column of water. In some other instances, the temperature sensor data may be received and/or recorded at one or more depths while the castable device 204B is holding or remains stationary in the column of water.

In some instances, fish may seek out and hold in water columns at particular temperature levels, e.g., particularly in temperature breaks where water temperature may rapidly change. In these instances, searching for these temperature breaks may assist anglers and fishermen with detecting active fish and productive water depths for fishing. Therefore, the castable device 204B with one or more environmental sensors 210A, 210B, . . . , 210N may assist anglers and fishermen with targeting different temperature zones to possibly increase fishing productivity.

In various implementations, at least one of the environmental sensors 210A, 210B, . . . , 210N may include a light sensor configured to detect, determine, and/or identify light intensity at one or more depths of the castable device 204B in a column of water. Further, the light sensor may be configured to generate sensor data including light intensity data corresponding to depth data at the one or more depths of the castable device 204B. In some instances, the light intensity sensor data may be received and/or recorded at a surface of a column of water. In other instances, the light intensity sensor data may be received and/or recorded at one or more depths during vertical movement (e.g., ascent and/or descent) of the castable device 204B in a column of water. In some other instances, the light intensity sensor data may be received and/or recorded at one or more depths while the castable device 204B is holding or remains stationary in the column of water.

In various implementations, at least one of the environmental sensors 210A, 210B, . . . , 210N may include an electrolyte sensor configured to detect, determine, and/or identify electrolyte concentration at one or more depths of the castable device 204B in a column of water. Further, the electrolyte sensor may be configured to generate sensor data including electrolyte concentration data corresponding to depth data at the one or more depths of the castable device 204B. In some instances, the electrolyte sensor data may be received and/or recorded at a surface of a column of water. In other instances, the electrolyte sensor data may be received and/or recorded at one or more depths during vertical movement (e.g., ascent and/or descent) of the castable device 204B in a column of water. In some other instances, the electrolyte sensor data may be received and/or recorded at one or more depths while the castable device 204B is holding or remains stationary in the column of water.

In various implementations, at least one of the environmental sensors 210A, 210B, . . . , 210N may include an acidity sensor configured to detect, determine, and/or identify acidity or acid concentration at one or more depths of the castable device 204B in a column of water. Further, the acidity sensor may be configured to generate sensor data including acidity or acid concentration data corresponding to depth data at the one or more depths of the castable device 204B. In some instances, the acidity sensor data may be received and/or recorded at a surface of a column of water. In other instances, the acidity sensor data may be received and/or recorded at one or more depths during vertical movement (e.g., ascent and/or descent) of the castable device 204B in a column of water. In still some other instances, the acidity sensor data may be received and/or recorded at one or more depths while the castable device 204B is holding or remains stationary in the column of water.

In various implementations, the one or more environmental sensors 210A, 210B, . . . , 210N may include a water sensor. Therefore, in some instances, the castable device 120 may include electrode terminals as part of a water sensor configured to activate the castable device 204B when the castable device 204B is deployed in water (e.g., the body of water 102). In this instance, the water sensor may be configured for automatically sensing deployment of the castable device 204B in water, which may occur after casting the castable device 204B in water by a user. Further, the electrode terminals of the water sensor may be configured to deactivate the castable device 204B when the castable device 204B is removed from water. In some instances, the memory instructions may be configured to cause the processor to transmit sensor data in response to the castable device 204B surfacing from water or detecting removal from water. In other instances, the water sensor may be configured to automatically detect or sense removal of the castable device 204B from water, which may occur after reeling in the castable device 204B out of water by a user.

In various implementations, the computing device 240 may be configured as a special purpose machine for interfacing with a castable device 204B having multiple environmental sensors 210A, 210B, . . . , 210N. The computing device 240 may include standard elements and/or components, including the at least one processor 242, the memory 244 (e.g., non-transitory computer-readable storage medium), at least one database 280, power, peripherals, and various other computing components that may not be specifically shown in FIG. 2A. Further, the computing device 240 may include the display device 270 (e.g., a monitor or other computer display) that may be used to provide a user interface (UI) 272, including a graphical user interface (GUI). In FIG. 2A, the display 270 is shown as an incorporated part of the computing device 240; however, the display 270 may be implemented as a separate component. Further, the UI 272 may be used to receive one or more preferences from a user of the display device 270 for managing or utilizing the sensor system 200A, including interfacing with the castable device 204A and the one or more environmental sensors 210. As such, a user may setup desired behavior of the sensor system 200A and/or the one or more environmental sensors 210 via user-selected preferences using the UI 272 associated with the display device 270. Various elements and/or components of the system 200A that may be useful for the purpose of implementing the system 200A may be added, included, and/or interchanged, in manner as described herein.

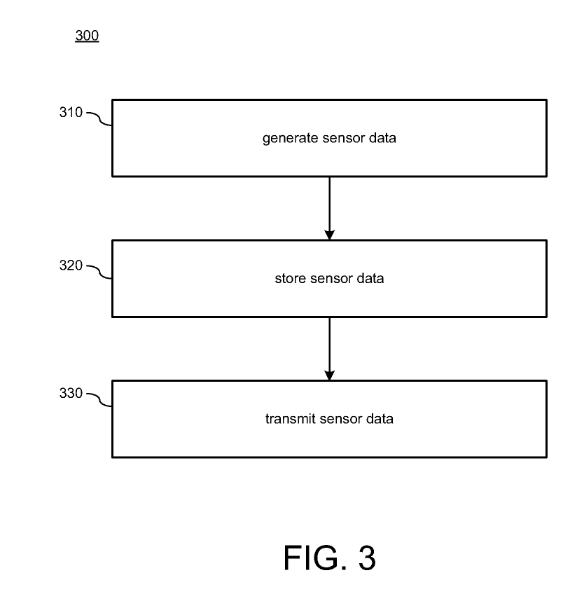

FIG. 3 illustrates a process flow diagram for a method 300 performed by a castable sensor device in accordance with implementations of techniques described herein. It should be understood that while method 300 indicates a particular order of execution of operations, in some instances, certain portions of the operations may be executed in a different order, and on different systems. Further, in some other instances, additional operations or steps may be added to method 300. Similarly, some operations or steps may be omitted.

At block 310, method 300 may generate sensor data. For instance, method 300 may be performed by a castable sensor device, which includes one or more environmental sensors configured to generate sensor data at one or more depths of the device in a column of water. As described herein, the one or more environmental sensors may be configured to detect, determine, and/or identify levels of environmental content, concentrations, and/or characteristics of the column of water at a surface and/or at various depths beneath the surface. In various instances, the environmental sensors may be configured to generate sensor data related to depth, pressure, chlorophyll concentration, oxygen concentration, temperature, light intensity, electrolyte concentration (e.g., salt content), acidity, etc.

At block 320, method 300 may store sensor data. For instance, method 300 may store, record, and/or log sensor related data generated by one or more environmental sensors at various depths in a column of water.

At block 330, method 300 may transmit sensor data. For instance, method 300 may transmit sensor data generated by one or more environmental sensors. In some instances, method 300 may transmit sensor data recorded in memory. In some other instances, method 300 may transmit sensor data to a computing device for storing in a database and/or for display on a display component or device.

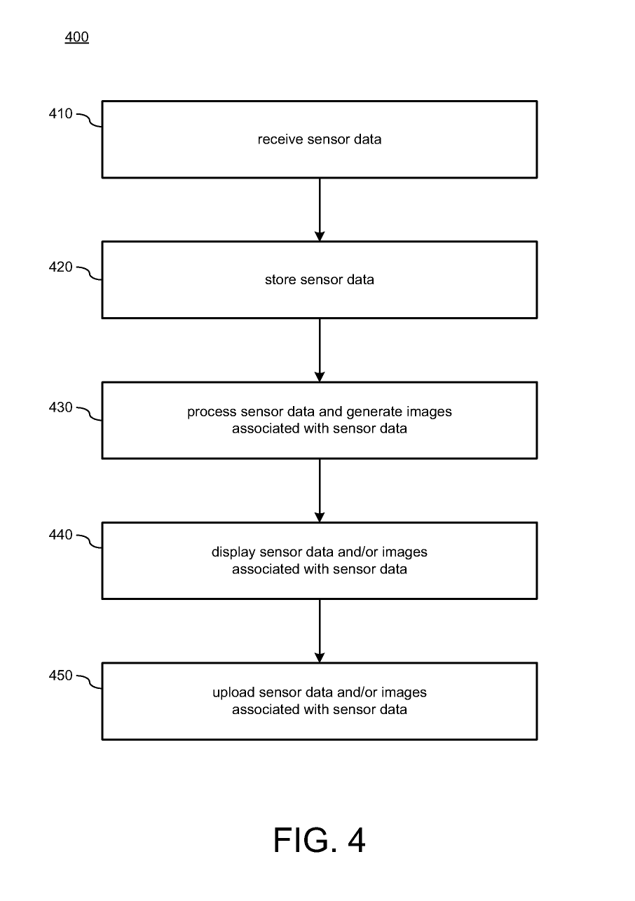

FIG. 4 illustrates a process flow diagram for a method 400 of using and/or operating a castable sensor device in accordance with implementations of techniques described herein. In one implementation, method 400 may be performed by a computing device in communication with a castable sensor device. It should be understood that while method 400 indicates a particular order of execution of operations, in some instances, certain portions of the operations may be executed in a different order, and on different systems. Further, in some other instances, additional operations or steps may be added to method 400. Similarly, some operations or steps may be omitted.

At block 410, method 400 may receive sensor data. For instance, the sensor data may be received from a castable sensor device via wired or wireless network. As described herein, the received sensor data may refer to various types of environmental data including, e.g., environmental sensor data related to depth, pressure, chlorophyll concentration, oxygen concentration, temperature, light intensity, electrolyte concentration (e.g., salt content), acidity, etc. Further, in some instances, method 400 may receive sensor data generated by one or more environmental sensors at various depths in a column of water.

At block 420, the sensor data received at block 410 may be stored. For instance, the sonar data may be stored by a computing device, e.g., the marine electronics device, configured to store, record, and/or log sensor related data received from a castable sensor device as generated by one or more environmental sensors at various depths in a column of water.

At block 430, method 400 may process sensor data and generate images associated with sensor data, and further at block 440, method 400 may display images associated with sensor data. For instance, the computing device may include a processor and memory having instructions that cause the processor to process sensor data including generating images associated with the sensor data. The images may include chart data, such as, e.g., chart data related to various environmental conditions of a column of water at various depths. The charted environmental data may include various types of environmental data including, e.g., environmental sensor data related to depth, pressure, chlorophyll concentration, oxygen concentration, temperature, light intensity, electrolyte concentration (e.g., salt content), acidity, etc. Further, the memory may include instructions that cause the processor to display images associated with the sensor data on a display component or device.

At block 450, method 400 may upload sensor data and/or images associated with sensor data to a network server. For instance, method 400 may be performed by a computing device configured to upload sensor data and/or images associated with sensor data to a network server.

### Computing System

Implementations of various technologies described herein may be operational with numerous general purpose or special purpose computing system environments or configurations. Examples of well-known computing systems, environments, and/or configurations that may be suitable for use with the various technologies described herein include, but are not limited to, personal computers, server computers, hand-held or laptop devices, multiprocessor systems, microprocessor-based systems, set top boxes, programmable consumer electronics, network PCs, minicomputers, mainframe computers, smart phones, tablets, wearable computers, cloud computing systems, virtual computers, marine electronics devices, and the like.

The various technologies described herein may be implemented in the general context of computer-executable instructions, such as program modules, being executed by a computer. Generally, program modules include routines, programs, objects, components, data structures, etc. that perform particular tasks or implement particular abstract data types. Further, each program module may be implemented in its own way, and all need not be implemented the same way. While program modules may all execute on a single computing system, it should be appreciated that, in some implementations, program modules may be implemented on separate computing systems or devices adapted to communicate with one another. A program module may also be some combination of hardware and software where particular tasks performed by the program module may be done either through hardware, software, or both.

The various technologies described herein may be implemented in the context of marine electronics, such as devices found in marine vessels and/or navigation systems. Ship instruments and equipment may be connected to the computing systems described herein for executing one or more navigation technologies. The computing systems may be configured to operate using various radio frequency technologies and implementations, such as sonar, radar, GPS, and like technologies.

The various technologies described herein may also be implemented in distributed computing environments where tasks are performed by remote processing devices that are linked through a communications network, e.g., by hardwired links, wireless links, or combinations thereof. In a distributed computing environment, program modules may be located in both local and remote computer storage media including memory storage devices.

### Marine Electronics Device

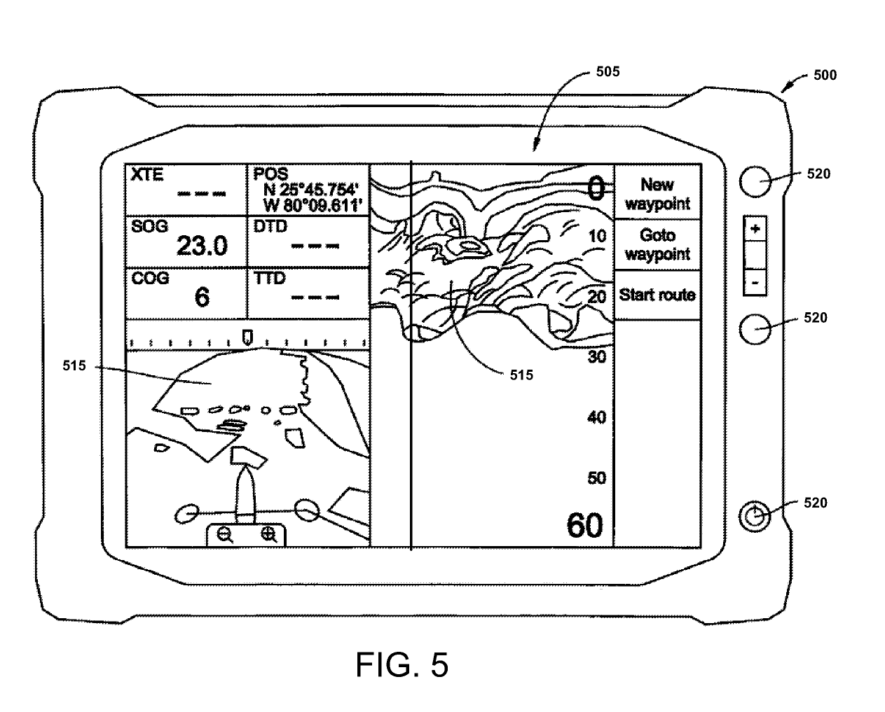

FIG. 5 illustrates an instance schematic of a marine electronics device 500 in accordance with implementations of various techniques described herein. The marine electronics device 500 includes a screen 505. In some instances, the screen 505 may be sensitive to touching by a finger. In other instances, the screen 505 may be sensitive to the body heat from the finger, a stylus, or responsive to a mouse. In various implementations, the marine electronics device 500 may be attached to various buses and/or networks, such as, e.g., a National Marine Electronics Association (NMEA) bus or network. The marine electronics device 500 may send or receive data to or from another device attached to the NMEA 2000 bus. For instance, the marine electronics device 500 may transmit commands and receive data from a motor or a sensor using an NMEA 2000 bus. In some implementations, the marine electronics device 500 may be capable of steering a vessel and controlling the speed of the vessel, i.e., autopilot. For instance, one or more waypoints may be input to the marine electronics device 500, and the marine electronics device 500 may be configured to steer the vessel to the one or more waypoints. Further, the marine electronics device 500 may be configured to transmit and/or receive NMEA 2000 compliant messages, messages in a proprietary format that do not interfere with NMEA 2000 compliant messages or devices, and/or messages in any other format. In various other implementations, the marine electronics device 400 may be attached to various other communication buses and/or networks configured to use various other types of protocols that may be accessed via, e.g., NMEA 2000, NMEA 0183, Ethernet, Proprietary wired protocol, etc.

The marine electronics device 500 may be operational with numerous general purpose or special purpose computing system environments and/or configurations. The marine electronics device 500 may include any type of electrical and/or electronics device capable of processing data (including, e.g., various environmental sensor type data) and information via a computing system. The marine electronics device 500 may include various marine instruments, such that the marine electronics device 500 may use the computing system to display and/or process the one or more types of marine electronics data. The device 500 may display marine electronic data 515, such as, e.g., sensor data and images associated with sensor data. The marine electronic data types 515 may include various chart data, radar data, sonar data, steering data, dashboard data, navigation data, fishing data, engine data, and the like. The marine electronics device 500 may include one or more buttons 520, which may include physical buttons or virtual buttons, or some combination thereof. The marine electronics device 500 may receive input through a screen 505 sensitive to touch or buttons 520.

In some implementations, according to various techniques described herein, the marine electronics device 500 may be configured to simultaneously display images associated with one or more environmental sensors, an array of environmental sensors, and the like. For instance, the marine electronics device 500 may be configured to simultaneously display images associated with a plurality of environmental sensors, including environmental sensor data related to various environmental conditions of a column of water, such as, e.g., depth, pressure, chlorophyll concentration, oxygen concentration, temperature, light intensity, electrolyte concentration (e.g., salt content), acidity, etc. In some instances, in various display modes of operation, the marine electronics device 500 may be configured to simultaneously display images associated with environmental sensor data on the screen 505. Further, environmental data related to detected environmental conditions of water may be displayed on the screen 505 of the marine electronics device 500 by overlaying various environmental data on chart and sonar images. In some instances, various environmental data may include current levels of environmental conditions at particular depths, upper and lower boundary levels at particular depths, average levels through a water column, and any changes that occur throughout sensor use during a particular time period or interval.

The marine electronics device 500 may be configured as a computing system having a central processing unit (CPU), a system memory, a graphics processing unit (GPU), and a system bus that couples various system components including the system memory to the CPU. In various implementations, the computing system may include one or more CPUs, which may include a microprocessor, a microcontroller, a processor, a programmable integrated circuit, or a combination thereof. The CPU may include an off-the-shelf processor such as a Reduced Instruction Set Computer (RISC), or a Microprocessor without Interlocked Pipeline Stages (MIPS) processor, or a combination thereof. The CPU may also include a proprietary processor.

The GPU may be a microprocessor specifically designed to manipulate and implement computer graphics. The CPU may offload work to the GPU. The GPU may have its own graphics memory, and/or may have access to a portion of the system memory. As with the CPU, the GPU may include one or more processing units, and each processing unit may include one or more cores.

The CPU may provide output data to a GPU. Further, the GPU may generate user interfaces (UIs) including graphical user interfaces (GUIs) that provide, present, and/or display the output data. The GPU may also provide objects, such as menus, in the GUI. In some instances, a user may provide input by interacting with objects, and the GPU may receive input from interaction with objects and provide the received input to the CPU. Further, in some instances, a video adapter may be provided to convert graphical data into signals for a monitor, such as, e.g., a multi-function display (MFD 500). The monitor (i.e., MFD 500) includes a screen 505. In various instances, the screen 505 may be sensitive to touch by a human finger, and/or the screen 505 may be sensitive to body heat from a human finger, a stylus, and/or responsive to a mouse.

The system bus may be any of several types of bus structures, including a memory bus or memory controller, a peripheral bus, and a local bus using any of a variety of bus architectures. By way of instance, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus. The system memory may include a read only memory (ROM) and a random access memory (RAM). A basic input/output system (BIOS), containing the basic routines that help transfer information between elements within the computing system, such as during start-up, may be stored in the ROM.

The computing system may further include a hard disk drive interface for reading from and writing to a hard disk, a memory card reader for reading from and writing to a removable memory card, and an optical disk drive for reading from and writing to a removable optical disk, such as a CD ROM or other optical media. The hard disk, the memory card reader, and the optical disk drive may be connected to the system bus by a hard disk drive interface, a memory card reader interface, and an optical drive interface, respectively. The drives and their associated computer-readable media may provide nonvolatile storage of computer-readable instructions, data structures, program modules and other data for the computing system.

Although the computing system is described herein as having a hard disk, a removable memory card and a removable optical disk, it should be appreciated by those skilled in the art that the computing system may also include other types of computer-readable media that may be accessed by a computer. For instance, such computer-readable media may include computer storage media and communication media. Computer storage media may include volatile and non-volatile, and removable and non-removable media implemented in any method or technology for storage of information, such as computer-readable instructions, data structures, program modules, software modules, or other data. Computer-readable storage media may include non-transitory computer-readable storage media. Computer storage media may further include RAM, ROM, erasable programmable read-only memory (EPROM), electrically erasable programmable read-only memory (EEPROM), flash memory or other solid state memory technology, CD-ROM, digital versatile disks (DVD), or other optical storage, magnetic cassettes, magnetic tape, magnetic disk storage or other magnetic storage devices, or any other medium which can be used to store the desired information and which can be accessed by the computing system. Communication media may embody computer readable instructions, data structures, program modules or other data in a modulated data signal, such as a carrier wave or other transport mechanism and may include any information delivery media. The term “modulated data signal” may mean a signal that has one or more of its characteristics set or changed in such a manner as to encode information in the signal. By way of instance, and not limitation, communication media may include wired media such as a wired network or direct-wired connection, and wireless media such as acoustic, radio frequency (RF), infrared (IR), and other wireless media. The computing system may include a host adapter that connects to a storage device via a small computer system interface (SCSI) bus, Fiber Channel bus, eSATA bus, or using any other applicable computer bus interface.

The computing system can also be connected to a router to establish a wide area network (WAN) with one or more remote computers. The router may be connected to the system bus via a network interface. The remote computers can also include hard disks that store application programs. In another implementation, the computing system may also connect to the remote computers via local area network (LAN) or the WAN. When using a LAN networking environment, the computing system may be connected to the LAN through the network interface or adapter. The LAN may be implemented via a wired connection or a wireless connection. The LAN may be implemented using Wi-Fi™′ technology, cellular technology, Bluetooth™ technology, satellite technology, or any other implementation known to those skilled in the art. The network interface may also utilize remote access technologies (e.g., Remote Access Service (RAS), Virtual Private Networking (VPN), Secure Socket Layer (SSL), Layer 2 Tunneling (L2T), or any other suitable protocol). In some instances, these remote access technologies may be implemented in connection with the remote computers. It will be appreciated that the network connections shown are exemplary and other means of establishing a communications link between the computer systems may be used.

A number of program modules may be stored on the hard disk, memory card, optical disk, ROM or RAM, including an operating system, one or more application programs, and program data. In certain implementations, the hard disk may store a database system. The database system could include, for instance, recorded points. The application programs may include various mobile applications (“apps”) and other applications configured to perform various methods and techniques described herein. The operating system may be any suitable operating system that may control the operation of a networked personal or server computer.

A user may enter commands and information into the computing system through input devices such as buttons, which may be physical buttons, virtual buttons, or combinations thereof. Other input devices may include a microphone, a mouse, or the like (not shown). These and other input devices may be connected to the CPU through a serial port interface coupled to system bus, but may be connected by other interfaces, such as a parallel port, game port or a universal serial bus (USB).

Certain implementations may be configured for connection to a GPS receiver system and/or a marine electronics device or system. The GPS system and/or marine electronics device or system may be connected via a network interface. For instance, the GPS receiver system may be used to determine position data for the vessel on which the marine electronics device 500 is disposed. Further, the GPS receiver system may transmit position data to the marine electronics device 500. In other instances, any positioning system known to those skilled in the art may be used to determine and/or provide the position data for the marine electronics device 500.

In various implementations, the marine electronics device or system may include one or more components disposed at various locations on a vessel. Such components may include one or more data modules, sensors, instrumentation, and/or any other devices known to those skilled in the art that may transmit various types of data to the marine electronics device 500 for processing and/or display. The various types of data transmitted to the marine electronics device 500 may include marine electronics data and/or other data types known to those skilled in the art. The marine electronics data received from the marine electronics device or system may include chart data, sonar data, structure data, radar data, navigation data, position data, heading data, automatic identification system (AIS) data, Doppler data, speed data, course data, or any other type known to those skilled in the art.

In one implementation, the marine electronics device or system may include a radar sensor for recording the radar data and/or the Doppler data, a compass heading sensor for recording the heading data, and a position sensor for recording the position data. In a further implementation, the marine electronics device or system may include a sonar transducer for recording the sonar data, an AIS transponder for recording the AIS data, a paddlewheel sensor for recording the speed data, and/or the like.

The marine electronics device 500 may receive external data via a LAN or a WAN. In some implementations, external data may relate to information not available from various marine electronics systems. The external data may be retrieved from the Internet or any other source. The external data may include atmospheric temperature, atmospheric pressure, tidal data, weather, temperature, moon phase, sunrise, sunset, water levels, historic fishing data, and/or various other fishing data.

In one implementation, the marine electronics device 500 may be a multi-function display (MFD) unit, such that the marine electronics device 500 may be capable of displaying and/or processing multiple types of marine electronics data. FIG. 5 illustrates a schematic diagram of an MFD unit in accordance with implementations of various techniques described herein. In particular, the MFD unit may include the computing system, the monitor (MFD 500), the screen 505, and the buttons such that they may be integrated into a single console.

The discussion of the present disclosure is directed to certain specific implementations. It should be understood that the discussion of the present disclosure is provided for the purpose of enabling a person with ordinary skill in the art to make and use any subject matter defined herein by the subject matter of the claims.

It should be intended that the subject matter of the claims not be limited to the implementations and illustrations provided herein, but include modified forms of those implementations including portions of the implementations and combinations of elements of different implementations within the scope of the claims. It should be appreciated that in the development of any such implementation, as in any engineering or design project, numerous implementation-specific decisions should be made to achieve a developers' specific goals, such as compliance with system-related and business related constraints, which may vary from one implementation to another. Moreover, it should be appreciated that such a development effort maybe complex and time consuming, but would nevertheless be a routine undertaking of design, fabrication, and manufacture for those of ordinary skill having benefit of this disclosure. Nothing in this application should be considered critical or essential to the claimed subject matter unless explicitly indicated as being “critical” or “essential.”

Reference has been made in detail to various implementations, instances of which are illustrated in the accompanying drawings and figures. In the following detailed description, numerous specific details are set forth to provide a thorough understanding of the present disclosure. However, the present disclosure may be practiced without these specific details. In other instances, well-known methods, procedures, components, circuits and networks have not been described in detail so as not to unnecessarily obscure aspects of the embodiments.

It should also be understood that, although the terms first, second, etc. may be used herein to describe various elements, these elements should not be limited by these terms. These terms are only used to distinguish one element from another. For instance, a first object or step could be termed a second object or step, and, similarly, a second object or step could be termed a first object or step, without departing from the scope of the invention. The first object or step, and the second object or step, are both objects or steps, respectively, but they are not to be considered the same object or step.

The terminology used in the description of the present disclosure herein is for the purpose of describing particular implementations and is not intended to limit the present disclosure. As used in the description of the present disclosure and appended claims, the singular forms “a,” “an,” and “the” are intended to include the plural forms as well, unless the context clearly indicates otherwise. The term “and/or” as used herein refers to and encompasses any and all possible combinations of one or more of the associated listed items. The terms “includes,” “including,” “comprises,” and/or “comprising,” when used in this specification, specify a presence of stated features, integers, steps, operations, elements, and/or components, but do not preclude the presence or addition of one or more other features, integers, steps, operations, elements, components and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context. As used herein, the terms “up” and “down”; “upper” and “lower”; “upwardly” and “downwardly”; “below” and “above”; and other similar terms indicating relative positions above or below a given point or element may be used in connection with some implementations of various technologies described herein.

While the foregoing is directed to implementations of various techniques described herein, other and further implementations may be devised without departing from the basic scope thereof, which may be determined by the claims that follow.

Although the subject matter has been described in language specific to structural features and/or methodological acts, it is to be understood that the subject matter defined in the appended claims is not necessarily limited to the specific features or acts described above. Rather, the specific features and acts described above are disclosed as instance forms of implementing the claims.

What is claimed is:

1. A device, comprising:
an environmental sensor configured to generate sensor data at one or more depths of the device in a body of water;
a processor; and
memory having instructions that cause the processor to:
receive the sensor data from the environmental sensor in response to the device being deployed at the one or more depths in the body of water; and
record the sensor data.

2. The device of claim 1, wherein the instructions further cause the processor to transmit the sensor data when the device surfaces from the body of water.

3. The device of claim 1, wherein the environmental sensor is configured to periodically generate the sensor data at pre-determined time intervals.

4. The device of claim 1, wherein the environmental sensor is configured to generate the sensor data during a vertical movement of the device as the device is ascending or descending in the body of water.

5. The device of claim 1, wherein the environmental sensor is configured to periodically generate sensor data at pre-determined depth intervals.

6. The device of claim 1, wherein the device is encapsulated within a waterproof housing that is impervious to water.

7. The device of claim 1, wherein the environmental sensor comprises a pressure sensor configured to:
determine pressure at the one or more depths; and
generate the sensor data having pressure data corresponding to the one or more depths.

8. The device of claim 1, wherein the environmental sensor comprises a chlorophyll sensor configured to:
determine chlorophyll concentration at the one or more depths; and
generate the sensor data having chlorophyll concentration data corresponding to the one or more depths.

9. The device of claim 1, wherein the environmental sensor comprises an oxygen sensor configured to:
determine oxygen concentration at the one or more depths, and
generate the sensor data having oxygen concentration data corresponding to the one or more depths.

10. The device of claim 1, wherein the environmental sensor comprises a temperature sensor configured to:
determine temperature at the one or more depths; and
generate the sensor data having temperature data corresponding to the one or more depths.

11. The device of claim 1, wherein the environmental sensor comprises a light sensor configured to:
determine light intensity at the one or more depths; and
generate the sensor data having light intensity data corresponding to the one or more depths.

12. The device of claim 1, wherein the environmental sensor comprises an electrolyte sensor configured to:
determine electrolyte concentration at the one or more depths; and
generate the sensor data having electrolyte concentration data corresponding to the one or more depths.

13. The device of claim 1, wherein the environmental sensor comprises an acidity sensor configured to:
determine acidity at the one or more depths; and
generate the sensor data having acidity data corresponding to the one or more depths.

14. A castable lure, comprising:
a first sensor configured to determine water pressure at one or more depths of the castable lure in a body of water, and generate depth data based on the determined water pressure at the one or more depths of the castable lure in the body of water;
a second sensor configured to generate environmental data corresponding to the depth data at the one or more depths of the castable lure in the body of water; and
a computing component configured to record the first sensor data generated by the first sensor and record the second sensor data generated by the second sensor.

15. The castable lure of claim 14, wherein the computing component is configured to transmit the first and second sensor data when the device surfaces from the body of water.

16. The castable lure of claim 14, wherein the first and second sensors are configured to periodically generate the sensor data at pre-determined time or depth intervals during at least one of ascent and descent of the castable lure in the body of water.

17. The castable lure of claim 14, wherein the second sensor comprises at least one of:
a chlorophyll sensor configured to generate chlorophyll concentration data corresponding to the depth data at the one or more depths of the castable lure in the body of water,
an oxygen sensor configured to generate oxygen concentration data corresponding to the depth data at the one or more depths of the castable lure in the body of water,
a temperature sensor configured to generate temperature data corresponding to the depth data at the one or more depths of the castable lure in the body of water,
a light sensor configured to generate light intensity data corresponding to the depth data at the one or more depths of the castable lure in the body of water,
an electrolyte sensor configured to generate electrolyte concentration data corresponding to the depth data at the one or more depths of the castable lure in the body of water, and
an acidity sensor configured to generate acidity concentration data corresponding to the depth data at the one or more depths of the castable lure in the body of water.

18. A device, comprising:
a castable housing impervious to water;
one or more environmental sensors configured to generate sensor data at one or more depths in the water;
a processor; and
memory including instructions that cause the processor to:
record the sensor data generated by the one or more environmental sensors at the one or more depths in the water, and
transmit the sensor data in response to detecting a network.

19. The device of claim 18, wherein the network comprises a wired or wireless network, and wherein the castable device is configured to communicate with a computing device via the wired or wireless network.

20. The device of claim 18, wherein the one or more environmental sensors are configured to periodically generate the sensor data at pre-determined time or depth intervals during at least one of ascent and descent of the castable device in the body of water.
